(19) United States
(12) Patent Application Publication
(10) Pub. No.: US 2008/0262621 A1
(43) Pub. Date: Oct. 23, 2008

(54) I-BEAM SPACER

(75) Inventor: Josef Gorek, Ross, CA (US)

Correspondence Address: CARTER, DELUCA, FARRELL & SCHMIDT, LLP, 445 BROAD HOLLOW ROAD, SUITE 225, MELVILLE, NY, 11747, US

(73) Assignee: K2M, Inc., Leesburg, VA (US)

(21) Appl. No.: 12/104,528

(22) Filed: Apr. 17, 2008

Related U.S. Application Data
(60) Provisional application No. 60/925,107, filed on Apr. 17, 2007.

(51) Int. Cl. A61F 2/44

(52) U.S. Cl. 623/17.16

(57) ABSTRACT

An I-beam spacer for intervertebral fusion comprises a first flange and a second flange. The first and second flanges are spaced apart from one another and have planar structures. In addition, the first and second flanges include a first outer surface and a second outer surface transverse to the support member. The first and second outer surfaces face away from each other. Also, the first and the second flanges have a first inner surface and a second inner surface transverse to the support member, wherein the first and second inner surface face toward each other. A support member is positioned between the first flange and the second flange. The support member is transverse to the first flange and the second flange. A space is formed between the first flange and the second flange. This space is adapted to receive a bone support matrix.

### CROSS-REFERENCE TO RELATED APPLICATIONS

This application claims priority to, and the benefit of, U.S. Provisional Patent Application Ser. No. 60/925,107, filed on Apr. 17, 2007, which is hereby incorporated by reference in its entirety.

### BACKGROUND

1. Technical Field

The present disclosure relates generally to spinal stabilization devices and procedures. More particularly, the present disclosure relates to an I-beam spacer for use in spinal stabilization.

2. Background of Related Art

Intervertebral disks can degenerate over time. In some instances, the disk material is simply diseased. These unfortunate occurrences may lead to, among other things, a reduction in normal intervertebral height. In addition, degenerated or diseased intervertebral disks abnormally compress an opposing disk when the disk material is diseased. This unusual compression often results in persistent pain.

Doctors and scientists have developed several techniques to alleviate the pain caused by diseased intervertebral disk material. For instance, stabilization or arthrodesis of the intervertebral joint may reduce the pain associated with movement of an intervertebral joint having diseased disk material. These techniques, also generally known as spinal or joint fusion, entail removing the disk material that separates opposing vertebra and packing the void area with a suitable bone support matrix. The matrix fuses with the bone material of the vertebra, thereby joining the two opposing vertebra together.

Joint fusion typically involves the use of a fusion device commonly known as a spinal cage or an I-beam spacer. During fusion procedures, surgeons place a spinal cage in a recess formed between opposing vertebra. This recess usually extends through the cortical end plates of this vertebra. Most spinal cages, as well as other fusion devices, have a chamber, or another kind of suitable space, where bone chips, bone slurry, bone allograft, or any other suitable bone support matrix is placed for facilitating bony union between the vertebrae. Ultimately, this bony union promotes stabilization of vertebrae.

Many fusion devices are relatively large and occupy a significant area between opposing vertebrae. While this arrangement may provide proper spinal stabilization, it also has it drawbacks. For instance, relatively large fusion devices require removal of important vertebral structures and segments. These structures enhance proper spinal stabilization. The removal of these structures could cause an improper or undesirable lordosis.

Accordingly, there is a need for improved intervertebral stabilizing devices and methods. The present disclosure relates to a method and devices addressing these needs.

### SUMMARY

The present disclosure relates to I-beam spacers for spinal fusion. An embodiment of the I-beam spacer includes a first flange, a second flange, and a support member. The flange includes a first outer surface and a first inner surface. The first outer surface has a first central opening and at least one arc-shaped opening following a portion of a curvature of the first flange. Like the first flange, the second flange includes a second outer surface and a second inner surface. The second outer surface has a second central opening. The first outer surface and the second outer surface face away from each other and the first inner surface and the second outer surface face toward each other. The support member interconnects the first and second flanges and is positioned transversely with respect to the first and second flanges. A lumen extends through the support member such that the first central opening communicates with the second central opening, and is adapted to receive a bone support matrix for promoting bone growth between opposing vertebrae.

In another embodiment, the support member may include at least one opening extending across its width. In this embodiment, the first and second flanges are generally elongate structures. The inner surfaces of the flanges are substantially parallel to one another. The outer surfaces have a proximal portion, an apex or intermediate portion, and a distal portion. The distal portion extends posteriorly from the apex towards the distal end and is angled towards the inner surface. The proximal portion extends anteriorly from the apex and is angled towards the inner surface.

In an alternate embodiment, an I-beam spacer additionally includes a convex structure positioned at a distal end thereof. The convex structure has a pair of projecting edges extending distally therefrom. These projection edges are positioned between the first and second flanges and have substantially arcuate shapes. In this embodiment, the support member may contain at least one bore extending therethrough.

The present disclosure also relates to another embodiment of an I-beam spacer having a curved profile. The first and second flanges of this embodiment may include a plurality of bores disposed thereon. The bores are arranged in curved rows that run along the periphery of each flange. Thus, the rows of bores follow the curvature of first and second flange respectively.

Additionally, the present disclosure relates to an intervertebral fusion kit. The intervertebral fusion kit includes one or more of the presently disclosed I-beam spacers along with one or more insertion tools.

### BRIEF DESCRIPTION OF THE FIGURES

Embodiments of the presently disclosed I-beam spacer are described herein with reference to the accompanying drawings, wherein:

FIG. 1 is a perspective view of an I-beam spacer in accordance with an embodiment of the present disclosure;

FIG. 2 is a side plan view of the I-beam spacer of FIG. 1;

FIG. 3 is a bottom view of the I-beam spacer of FIG. 1;

FIG. 4 is a rear perspective view of an I-beam spacer in accordance with an embodiment of the present disclosure;

FIG. 5 is a side plan view of the I-beam spacer of FIG. 4;

FIG. 6 is top view of the I-beam spacer of FIG. 4;

FIG. 7 is a front view of the I-beam spacer of FIG. 4;

FIG. 8 is a front perspective view of an I-beam spacer in accordance with an embodiment of the present disclosure;

FIG. 9 is a front view of the I-beam spacer of FIG. 8;

FIG. 10 is a top view of the I-beam spacer of FIG. 8;

FIG. 11 is a side plan view of the I-beam spacer of FIG. 8;

FIG. 12 is a top cross-sectional view of the I-beam spacer of FIG. 9, taken along section line A-A of FIG. 1;

FIG. 13 is a front cross-sectional view of the I-beam spacer of FIG. 8 taken along section line B-B of FIG. 11;

FIG. 14 is a rear perspective view of an I-beam spacer in accordance with an embodiment of the present disclosure;

FIG. 15 is a front view of the I-beam spacer of FIG. 14;

FIG. 16 is a top view of the I-beam spacer of FIG. 14;

FIG. 17 is a side plan view of the I-beam spacer of FIG. 14;

FIG. 18 is a bottom cross-sectional view of the I-beam spacer of FIG. 14, taken along section line D-D of FIG. 17;

FIG. 19 is a front cross-sectional view of the I-beam spacer of FIG. 14, taken along section line E-E of FIG. 17;

FIG. 20 is a perspective view of an I-beam spacer in accordance with an embodiment of the present disclosure;

FIG. 21 is a top view of the I-beam spacer of FIG. 20;

FIG. 22 is a side plan view of the I-beam spacer of FIG. 20;

FIG. 23 is a front cross-sectional view of the I-beam spacer of FIG. 20, taken along section line H-H of FIG. 22;

FIG. 24 is a side plan view of a fork-shaped driver in accordance with an embodiment of the present disclosure;

FIG. 25 is top view of the fork-shaped driver of FIG. 24;

FIG. 26 is a top view of an elongated sleeve of the fork-shaped driver of FIG. 24;

FIG. 27 a side plan view of the elongated sleeve of FIG. 24;

FIG. 28 is a enlarged top view of a portion of the elongated sleeve of FIG. 24, taken around section A of FIG. 26;

FIG. 29 is a rear view of the elongated sleeve of FIG. 24;

FIG. 30 is a perspective view of a handle of the fork-shaped driver of FIG. 24;

FIG. 31 is a side plan view of the handle of FIG. 30;

FIG. 32 is a front view of the handle of FIG. 30;

FIG. 33 is a perspective view of a shaft of the fork-shaped drive of FIG. 24;

FIG. 34 is a side plan view of the shaft of FIG. 33;

FIG. 35 is a side sectional view of a portion of the shaft of FIG. 33, taken around section B of FIG. 34;

FIG. 36 is a side plan view of the fork-shaped driver of FIG. 24 with an I-beam spacer mounted on its distal end;

FIG. 37 is a top view of the fork-shaped driver of FIG. 24 with an I-beam spacer mounted on its distal end;

FIG. 38 is a perspective view of the fork-shaped drive of FIG. 24 with an I-beam spacer mounted on its distal end;

FIG. 39 is a perspective view of the forked-shaped driver of FIG. 24 with three I-beam spacers mounted on its distal end;

FIG. 40 is a top view of the forked-shaped driver of FIG. 24 with three I-beam spacers mounted on its distal end;

FIG. 41 is a side view showing an I-beam spacer positioned between two vertebrae;

FIG. 42 is a top view of three I-beam spacers arranged linearly in a recess of a vertical body; and

FIG. 43 is top view of four I-beam spacers positioned in a recess of a vertebral body.

### DETAILED DESCRIPTION

Embodiments of the presently disclosed I-beam spacer will now be described herein in detail with reference to the drawings in which like reference numerals identify similar or identical elements. As used herein, terms such as “above,” “below,” “forward,” “rearward,” etc. refer to the orientation of the figures or the direction of components and are simply used for convenience. In addition, a singular term generally includes the plural, and a plural term generally includes the singular unless otherwise indicated.

The present disclosure relates to devices and methods for use during intervertebral stabilization and arthrodesis procedures. Specifically, the presently disclosed fusion devices or I-beam spacers facilitate stabilization of intervertebral bodies. Typically, surgeons insert at least one of these I-beam spacers into a space formed between two opposing vertebra during intervertebral stabilization or arthrodesis procedures. The embodiments of the presently disclosed I-beam spacer do not occupy the entire space formed between opposing vertebrae. Rather, these I-beam spacers, for instance, are designed to fill only about one-third to one-half of this intervertebral space. Some I-beam spacers envisioned in the present disclosure are dimensioned to occupy less than one fifth of the volume encompassed by the intervertebral space. In any case, these I-beam spacers provide adequate spinal stabilization.

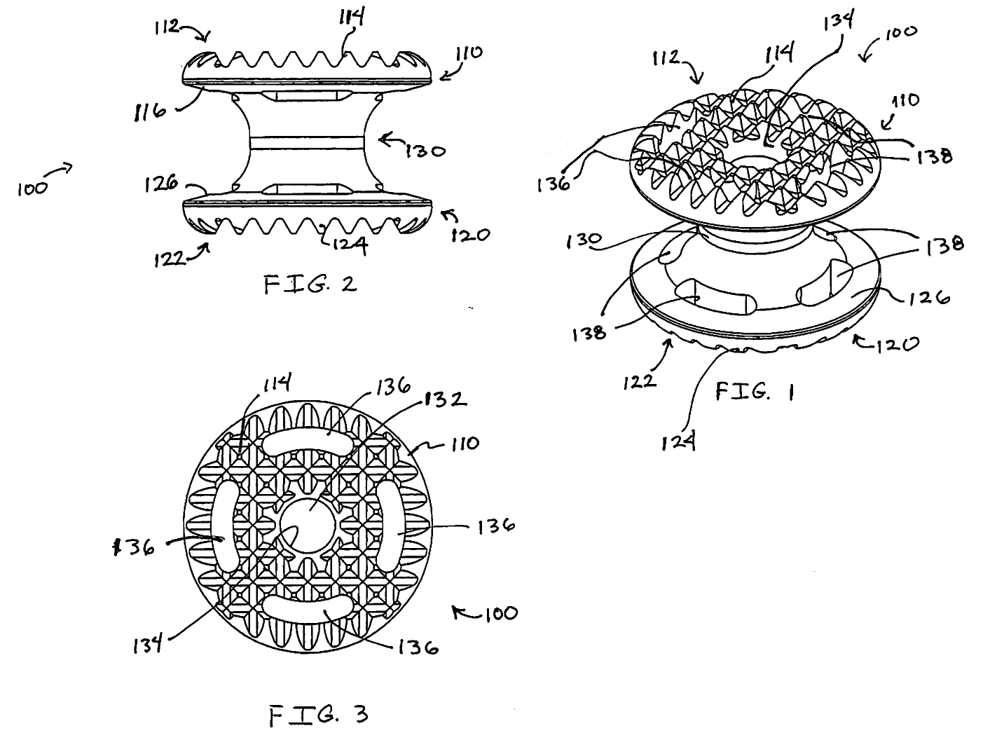

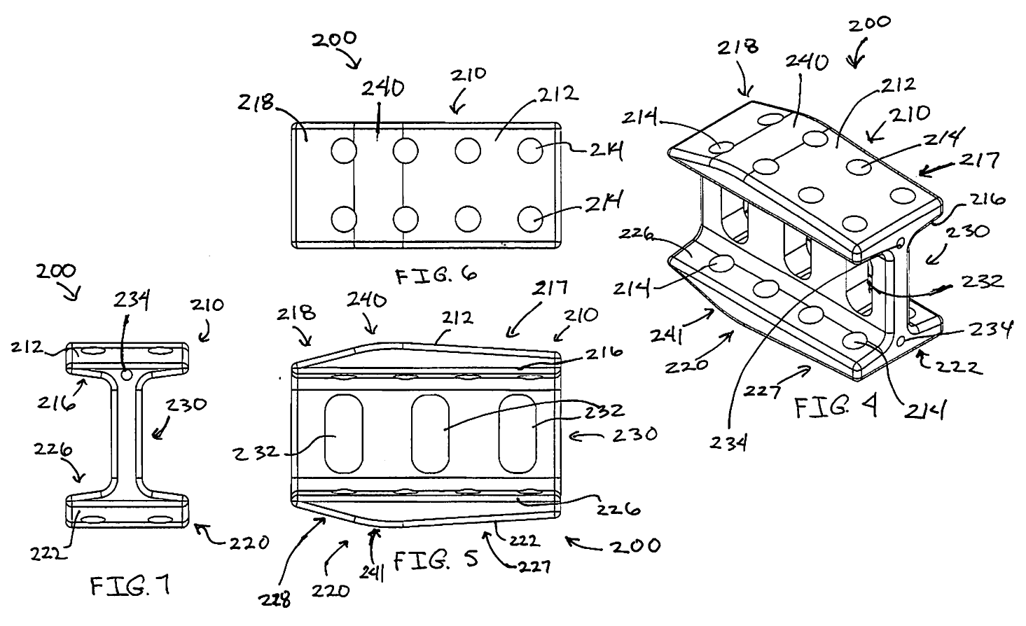

Referring to FIGS. 1-3, an embodiment of an I-beam spacer is generally identified with reference numeral 100. I-beam spacer 100 includes a first flange 110 and a second flange 120. In this embodiment, first flange 110 and second flange 120 have substantially circular shapes. The present disclosure, however, envisions I-beam spacers with additional flanges and/or other shapes. First flange 110 and second flange 120 are spaced apart from each other by a transverse support member 130. Although FIG. 1 illustrates a support member 130 positioned substantially orthogonal with respect to first and second flanges 110, 120, it is contemplated that support member 130 may be positioned in any manner insofar as it supports first flange 110 and second flange 120 therebetween. In addition, support member 130 defines a gap between first flange 110 and second flange 120.

In the embodiment shown in FIG. 1, support member 130 includes a lumen 132 extending therethrough and may have a substantially concave configuration. Lumen 132 has an inner wall 134 having a substantially concave structure. Although the drawings show an inner wall 134 having a substantially concave cylindrical shape, inner wall 134 of lumen 132 can have many different kinds of configurations, shapes, structures and dimensions. Alternatively, support member 130 may have a complete or substantially solid structure.

Lumen 132 of support member 130 is adapted to receive a suitable bone support matrix for promoting bone growth between opposing vertebra. Suitable bone support matrices may be resorbable or nonresorbable and osteoconductive or osteoinductive. Examples of suitable matrices include bone graft, synthetic materials, or any variety of bone morphogenic proteins (BMPs). Suitable bone support matrices also include heterologous, homologous, or autologous bone and derivates thereof. The bone support matrix can be radiolucent or radiopaque. Regardless of the specific bone support matrix employed, the selected bone support matrix material is disposed inside lumen 132. Bone support matrix may additionally be placed between first and second flanges 110, 120 to facilitate intervertebral bone growth.

First flange 110 and second flange 120 include opposing outer surfaces 112, 122, respectively. When positioned between the vertebrae, opposing outer surfaces 112, 122 contact and support the end plates of the vertebra. Outer surfaces 112, 122 are transverse to support member 130 and face away from one another. Additionally, first flange 110 and second flange 120 include opposing inner surfaces 116, 126 facing toward each other.

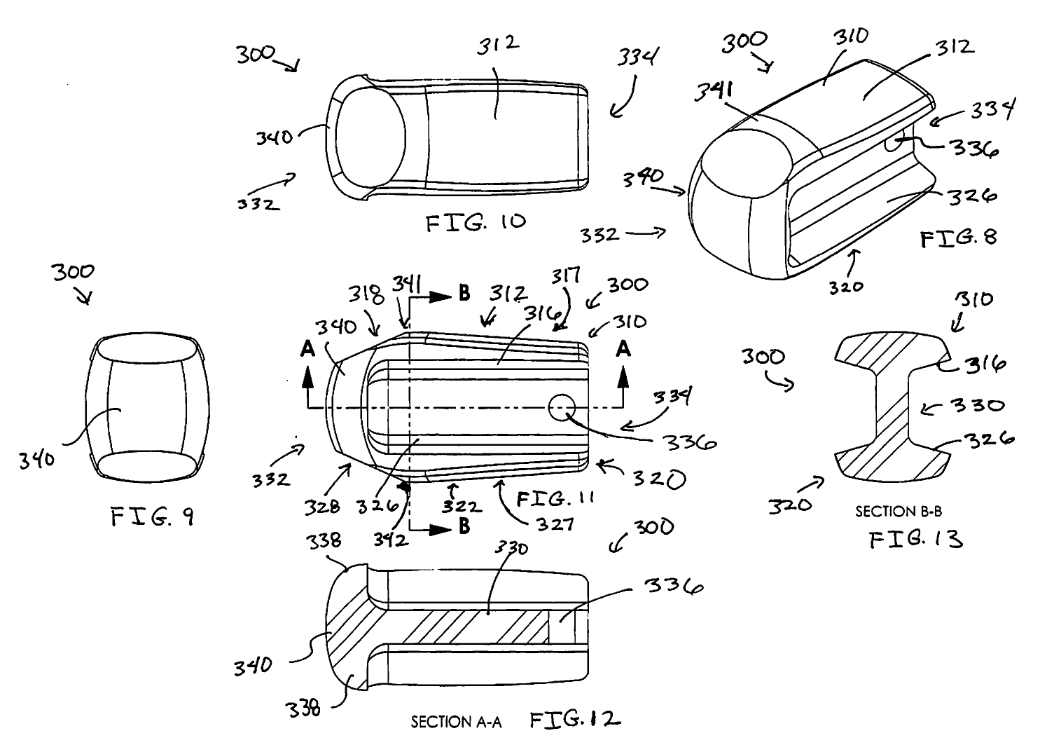

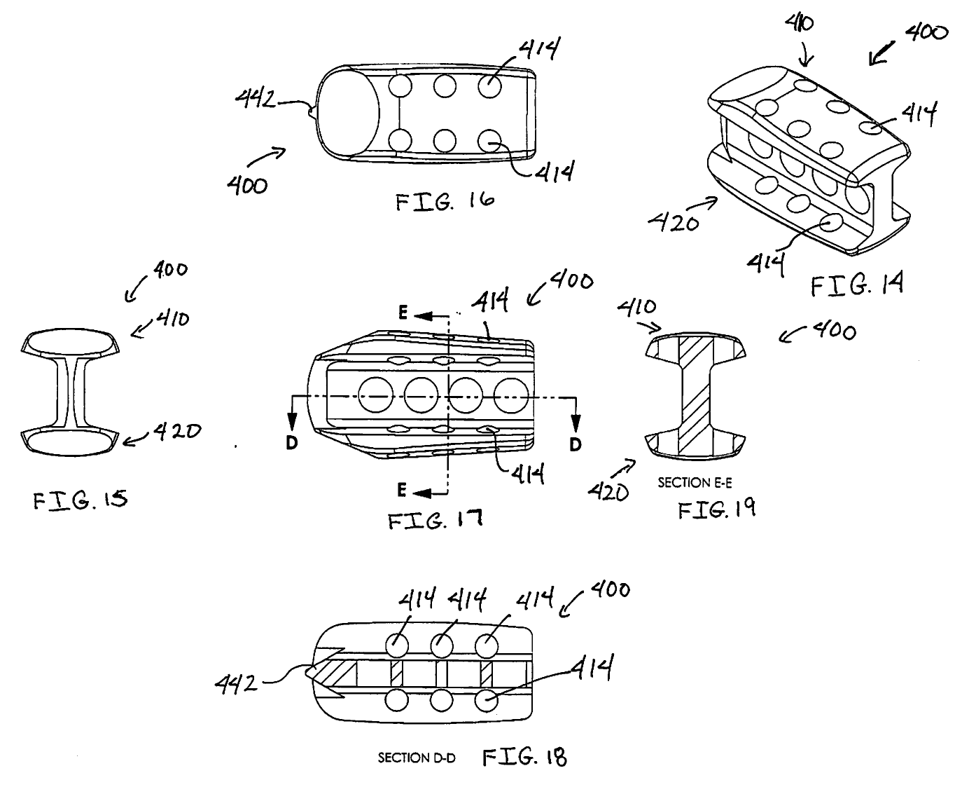

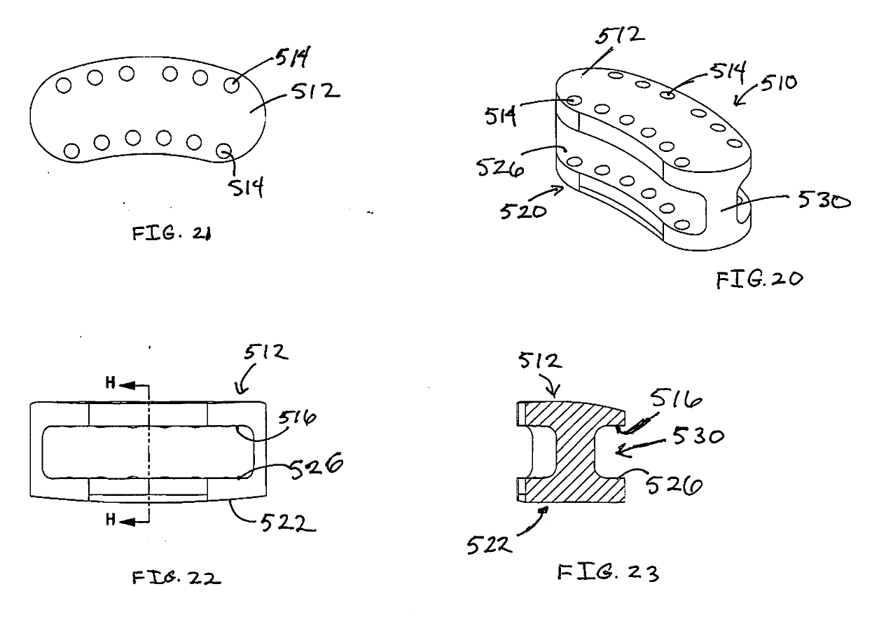

In the embodiment shown in FIGS. 1-3, outer surfaces 112, 122 have projections 114, 124 protruding outwardly therefrom. Projections 114, 124 may have pyramidal shapes, as shown in FIG. 1, or any other shape suitable for engaging the cortical plates of opposing vertebrae. As seen in FIGS. 2 and 3, projections 114, 124 are spaced apart from each other and spread across outer surfaces 112, 122 of first and second flanges 110, 120, respectively

The illustrated embodiment in FIGS. 1-3 also includes openings 136, 138 located along the periphery of first and second flanges 110, 120, respectively. Openings 136, 138 have substantial arcuate shapes following the curvature of outer surfaces 112, 122 and extend through first and second flanges 110, 120. Nonetheless, openings 136, 138 may be positioned in any suitable pattern and may have any appropriate shape or structure. Each flange 110, 120 may have four openings 136, 138 as shown in FIGS. 1 and 3. Nevertheless, one skilled in the art will recognize that one or more openings may be placed on first and second flanges 110, 120. In one embodiment, first and second flanges 110, 120 have indentations instead of openings. Still in another embodiment, first and second flanges 110, 120 include openings and indentations. Irrespective of the exact configuration of the I-beam spacer 100, the openings and indentations are designed to enhance bone growth between opposing vertebrae, thereby furthering spinal stabilization.

Referring to FIGS. 4-6, another embodiment of an I-beam spacer 200 includes a first flange 210, a second flange 220, and a support member 230. First flange 210 and second flange 220 are transverse to and spaced apart by support member 230. Support member 230 supports and interconnects first and second flanges 210, 220. In this embodiment, first and second flanges 210, 220 have substantially rectangular shapes.

First flange 210 and second flange 220 include opposing outer surfaces 212, 222, respectively, facing away from each other and opposing inner surfaces 216, 226, respectively, facing toward each other. Inner surfaces 216, 226 have substantially planar structures. In turn, each outer surface 212, 222 includes a proximal portion 217, 227 and a distal portion 218, 228. Proximal portions 217, 227 are located in a rear section of I-beam spacer 200, while the distal portions 218, 228 are positioned in a front section of I-beam spacer 200. Proximal portions 217, 227 are disposed at an angle with respect to inner surfaces 216, 226 such that the distance therebetween increases along the longitudinal axis of I-beam spacer 200 reaching a corresponding apex 240, 241 between the proximal and distal portions. Distal portions 218, 228 are angled with respect the inner surfaces 216, 226 such that the distance therebetween decreases along a portion of the longitudinal axis of I-beam spacer 200 that extends from the corresponding apex 240, 241 to the distal end of I-beam spacer 200.

A plurality of bores 214 extends through first flange 210 and second flange 220, respectively. In the embodiment shown in FIG. 3, bores 214 are arranged in longitudinal spaced apart rows. Specifically, the depicted embodiment includes two longitudinal spaced-apart rows of bores 214 in each flange 210, 220. The two rows of bores 214 on each flange 210, 220 are aligned with each other. Bores 214 also feature a circular cross-section. Those having ordinary skill in the art will contemplate bores having other shapes, orientations, and locations. For instance, bores 214 may have a substantially square-shaped cross-section. Bores 214 may also be arranged in two unaligned rows.

Support member 230 includes a plurality of spaced apart openings 232 having substantially elliptical shapes. Openings 232 extend throughout the entire width of support member 230. Additionally, support member 230 has two bores 234 extending through at least a portion of its length. Bore 234 has an opening on a front upper portion of support member 230, as shown in FIG. 7. Support member 230 may nevertheless have different configurations and structures. For example, a plurality of columns may form support member 230. Alternatively, I-beam spacer 200 may include a solid support member 230. This solid support member 230 may have indentations disposed thereon. In addition, openings 232 may have other suitable shapes.

Referring to FIGS. 8-13, an embodiment of an I-beam spacer 300 includes a support member 330, a first flange 310, and a second flange 320. Support member 330 interconnects first and second flanges 310, 320 and includes a bore 336 transverse to a longitudinal axis of the support member 330. Bore 336 features a substantially circular cross-section, but those with ordinary skill will recognize that bore 336 may have other suitable shapes. Further, bore 336 is located in a proximal portion of the support member 330. Support member 330, however, may include one or more bores in a plurality of alternate locations. Additionally, support member 330 is transverse to first and second flanges 310, 320.

First flange 310 and second flange 320 have opposing outer surfaces 312, 322 facing away from one another. In addition, first and second flanges 310, 320 include opposing inner surfaces 316, 326 facing toward one another. First and second flanges 310, 320 may have indentations or bores, or both, disposed thereon. Additionally, I-beam spacer 300 includes a front section 332 and a rear section 334. Front section 332 of the I-beam spacer 300 includes a convex structure 340 including a pair of projecting edges 338 extending outwardly therefrom. Projection edges 338 are positioned between first and second flanges 310, 320 and have substantially arcuate shapes.

As discussed above, first flange 310 and second flange 320 include opposing outer surfaces 312, 322 and opposing inner surfaces 316, 326. Inner surfaces 316, 326 are substantially planar structures. In turn, each outer surface 312, 322 includes a proximal portion 317, 327 and a distal portion 318, 328. Proximal portions 317, 327 are located are located in a rear section 334 of I-beam spacer 300, while the distal portions 318, 328 are positioned in a front section 332 of I-beam spacer 300. Proximal portions 317, 327 are arranged such that a gap defined between the inner surfaces 316, 326 is larger at an apex 341, 342 that at the rear section 334. Similarly, the distal portions 318, 328 converge from the apex 341, 342 towards the front section 332.

With reference to FIGS. 14-19, an alternate embodiment of the I-beam spacer 400 is substantially similar to the previously described I-beam spacer, 300. In this embodiment, I-beam spacer 400 has a pointed front section 442 that extends beyond the distal ends of the first and second flanges 410, 420. I-beam spacer 400 additionally includes a plurality of bores or holes 414 arranged in a pair of linear rows disposed on each flange 410, 420. Bores 414 allow bone growth therethrough and may have a substantially circular shape. In addition to bores 414, the support member 430 includes openings 432 extending therethrough. Openings 432 have a circular shape. The present disclosure, however, contemplates bores and openings having other shapes, orientations, and locations.

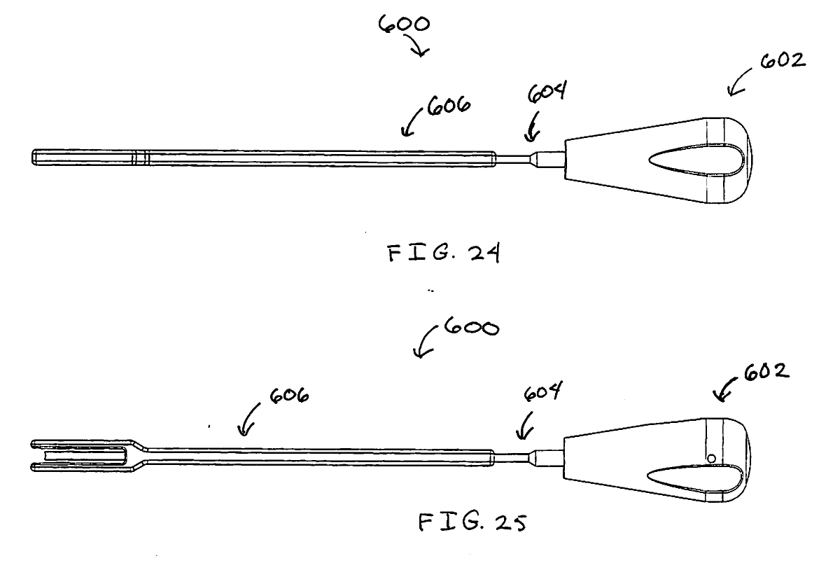

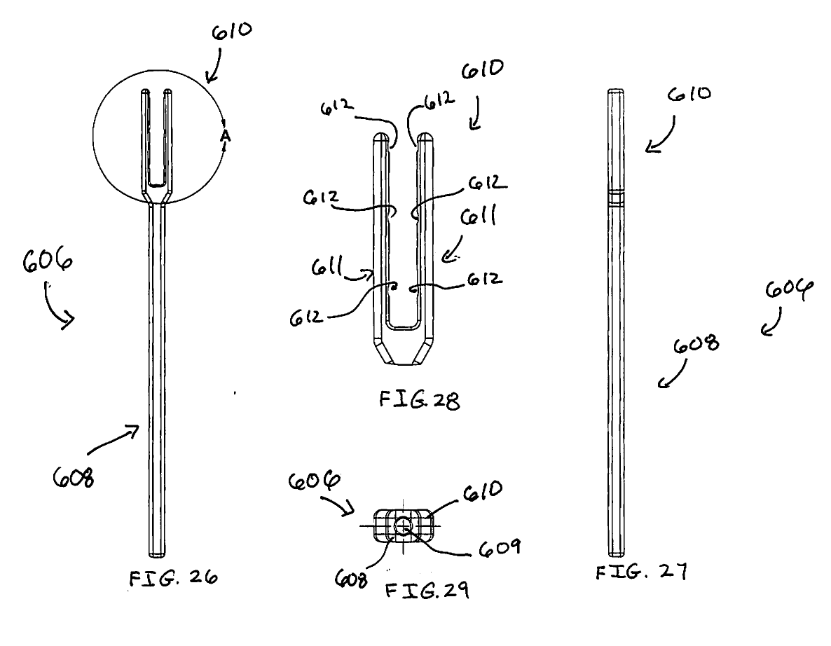

Referring to FIGS. 20-23, another embodiment of I-beam spacer 500 includes a support member 530, a first flange 510, and a second flange 520. Support member 530 interconnects and is transverse to first and second flanges 510, 520 and has concave exterior walls 532, 534. Specifically, I-beam spacer 500 is curved along its longitudinal axis defining a kidney bean shape. First flange 510 and second flange 520 have opposing outer surfaces 512, 522, respectively, facing away from one another. First and second flanges 510, 520 also include opposing inner surfaces 516, 526 facing toward one another. In this embodiment, first flange 510 and second flange 520 include a plurality of bores 514 disposed thereon. Bores 514 are arranged in curved rows that run along the periphery of each flange 510, 520 as seen in FIGS. 20 and 21. The rows of bores 514 follow the curvature of first and second flanges 510, 520. The present disclosure contemplates first and second flanges 510, 520 having various bore configurations and shapes. First and second flanges 510, 520 may alternatively have at least one indentation positioned thereon.

The embodiments of the presently disclosed I-beam spacer are made of polyether-ether-ketone (“PEEK”) or any other suitable material known in the art. Other suitable materials include titanium, titanium alloys, shape memory alloys, ceramics, composites, and stainless steel. Regardless of the material employed, the presently disclosed embodiments may have radiolucent properties.

Further, the present disclosure contemplates I-beam spacers having different sizes. For instance, an I-beam spacer used for the anterior portion of the vertebral disk could be larger than an I-beam spacer used for the posterior portion of the vertebral disk. Moreover, an embodiment of the disclosed I-beam spacer can be dimensioned to occupy from about a half to one third of the recess formed between opposing vertebrae. This specific embodiment may be placed close to the posterior portion of the vertebral disks.

In operation, the surgeons position any I-beam spacers disclosed herein in a recess formed between opposing vertebrae. To place I-beam in the desired location, the surgeon may utilize a surgical instrument such as the fork-shaped driver 600 shown in FIGS. 24-25. Fork-shaped driver 600 includes a handle 602 positioned at its proximal end 601, a tube or shaft 604 connected to the handle 602, and an elongated sleeve 606 positioned over the shaft 604.

As depicted in FIGS. 26-29, elongated sleeve 606 includes a tubular portion 608 and a holding member 610 adapted to retain an I-beam spacer. The tubular portion 606 has a lumen 609 extending therethrough and adapted to slidably receive the shaft 604. The holding member 610 has two prongs 611 spaced apart from each other. The two prongs 611 define a space therebetween dimensioned to receive an I-beam spacer. Prongs 611 extend in a distal direction with respect to the tubular portion 608 and each include at least one undulated surface 612 configured to receive a portion of an I-beam spacer. Together, a pair of undulated surfaces 612 in each prong 611 holds an I-beam spacer. In the embodiment shown in FIG. 28, holding member 610 includes three pairs of undulated surfaces 612. As a result, the depicted embodiment of driver 600 is capable of holding at least two I-beam spacers.

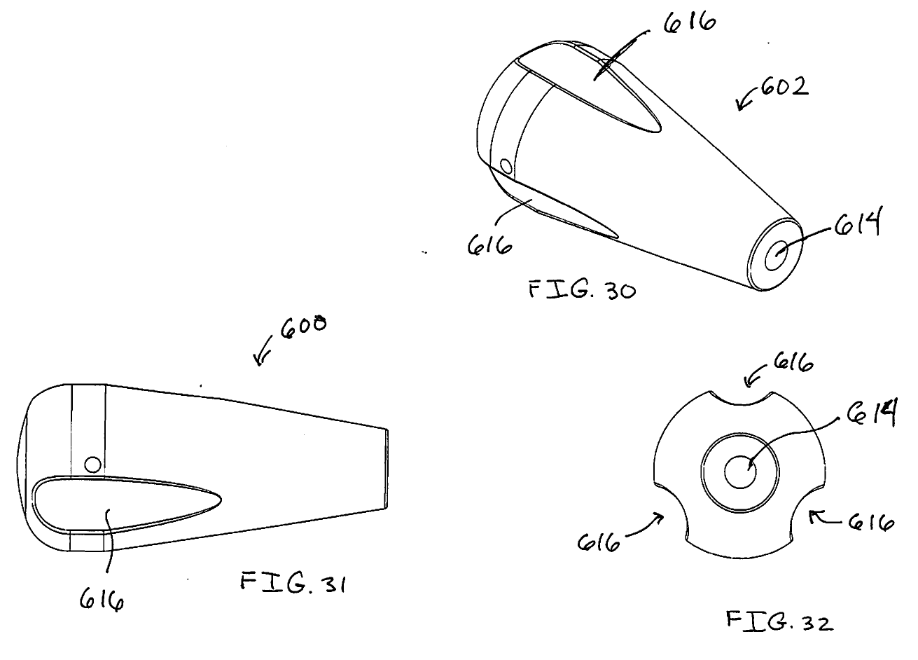

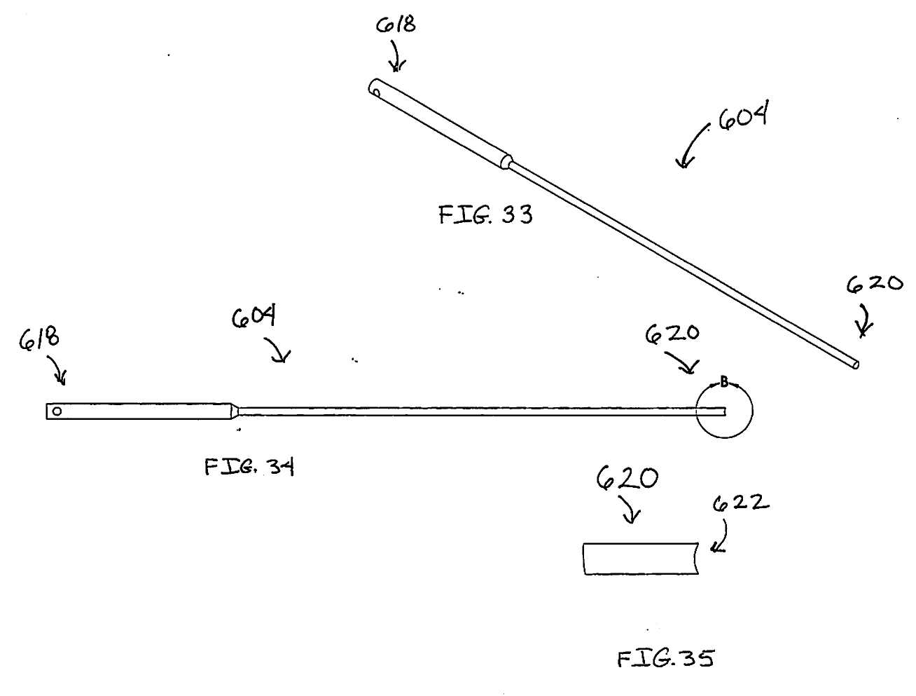

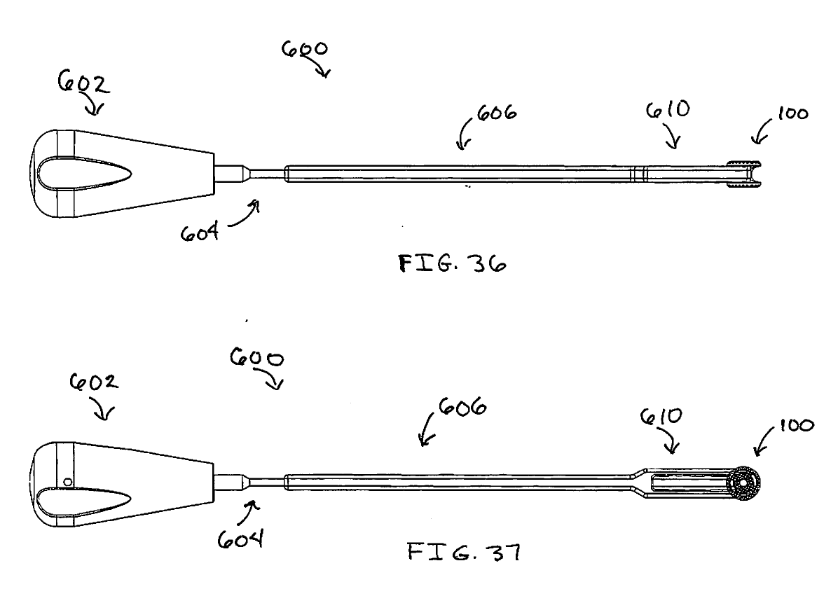

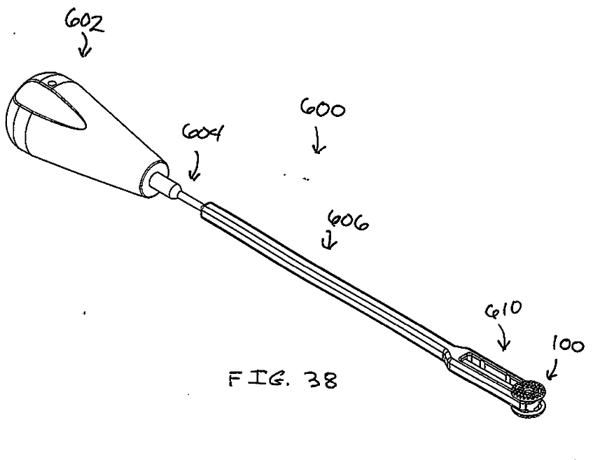

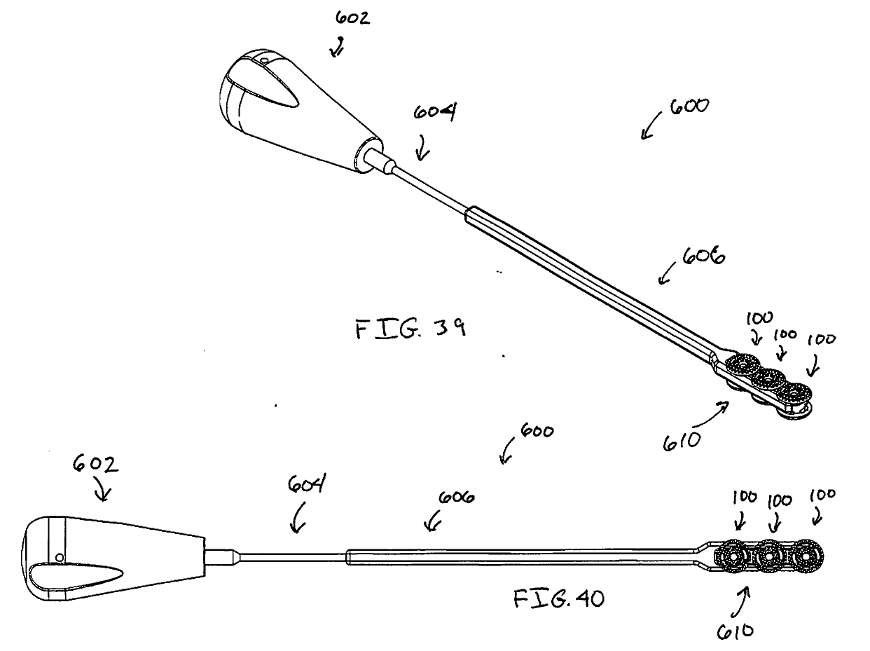

Referring to FIGS. 30-35, aside from the elongated sleeve 606, driver 600 contains a handle 602 as discussed above. Handle 602 includes indentations 616 adapted to receive a user's fingers and a bore 614 configured for securely receiving shaft 604. As seen in FIGS. 33-25, shaft 604 has a proximal end 618 configured for connection to the handle 602 and a distal end 620 adapted to receive at least a portion of an I-beam spacer. In one embodiment, as shown in FIG. 35, distal end 620 of shaft 604 includes a concave receiving surface 622 for driving an I-beam spacer toward the desired surgical site.

During spinal stabilization procedures, a doctor first prepares a recess between opposing vertebrae. Bone graft, BMP or any other suitable bone support matrix may be packed into the I-beam spacers' bores, indentations and/or open spaces between flanges. Thereafter, the doctor may use the fork-shaped driver 600 shown in FIGS. 36-38, or any other suitable apparatus, to place at least one I-beam spacer in the recess formed between two opposing vertebrae.

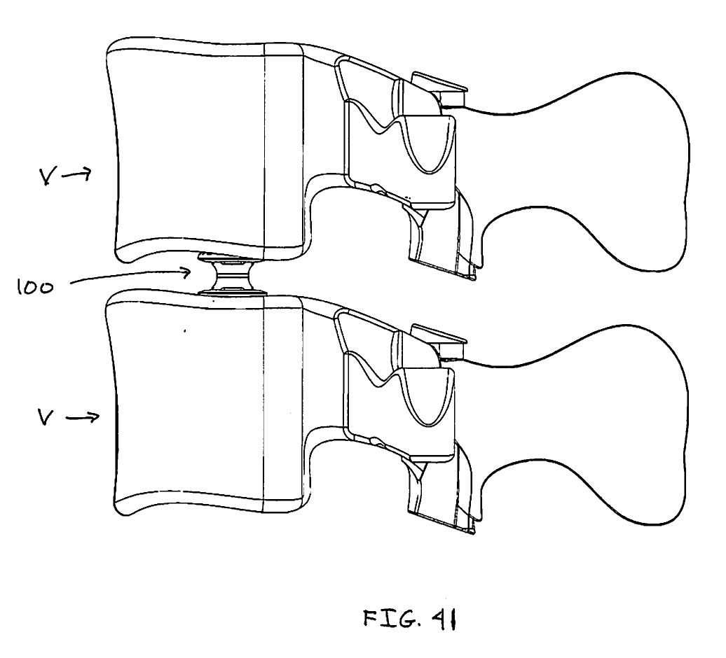

When the doctor employs the fork-shaped drive 600, the holding member 610 of the fork-shaped driver 600 holds one I-beam spacer, as seen in FIG. 38, or more than one I-beam spacer, as depicted in FIGS. 39 and 40. The holding member 610 retains I-beam spacers, when a doctor positions the support member of each I-beam spacer between a pair of undulated surfaces 612. Each pair of undulated surfaces 612 frictionally engages the support member of an I-beam spacer, thereby securing the I-beam spacer between the prongs 111 of the forked-shape driver 600.

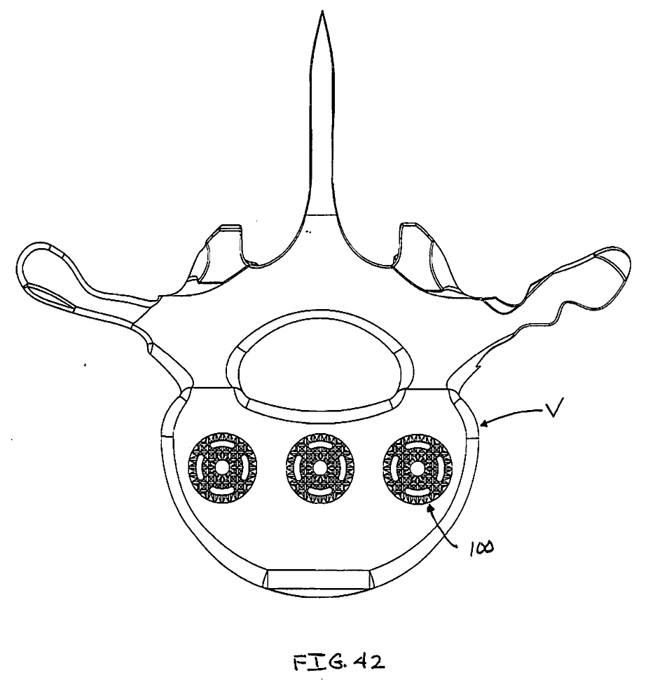

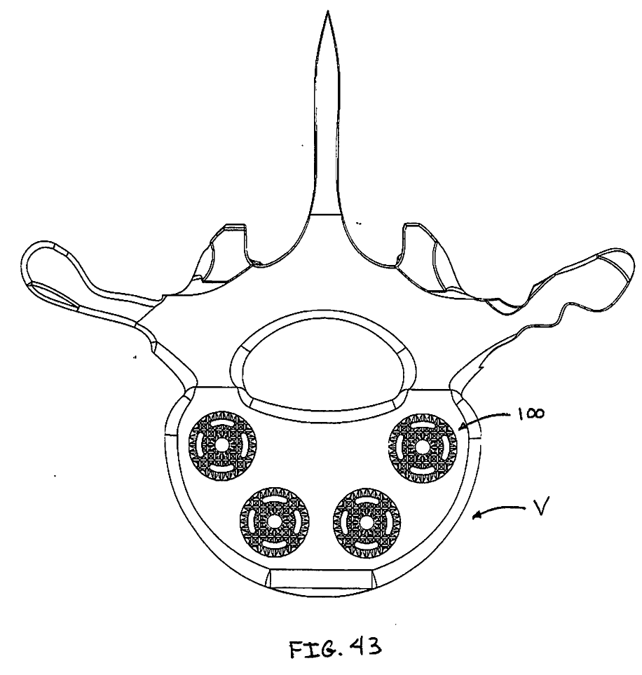

Once the I-beam spacers are properly positioned in the holding member 610 of the fork-shaped driver 600, the doctor places one or more I-beam spacers in the recess formed between the intervertebral bodies V, as illustrated in FIG. 41. The I-beam spacer may be arranged in numerous ways. For example, the doctor may position only one I-beam spacer in the recess. The remaining volume of the recess may be packed with bone support matrix. In an alternative arrangement, three I-beam spacers are placed in the recess. These I-beam spacers are arranged in a longitudinal row, as shown in FIG. 42. Bone support matrix packs the rest of the volume inside of the recess. Alternatively, the doctor may place four I-beam spacers in the recess, as seen in FIG. 43. These I-beams spacers are arranged in two unaligned rows. Bone support matrix fills the remaining volume of the recess. Irrespective of the number and arrangement of the I-beam spacers, the outer surfaces of the first and second flanges support the cortical end plates of the opposing vertebrae, thereby promoting spinal stabilization.

Those skilled in the art will understand that various modifications can be made to the embodiments disclosed herein. Therefore, the above description should not be construed as limiting, but merely as examples of embodiments. One having ordinary skills in the art will envision other modifications within the scope and spirit of the claims appended thereto.

What it is claimed is:

1. An I-beam spacer for intervertebral fusion, comprising:
a first flange including a first outer surface and a first inner surface;
a second flange including a second outer surface and a second inner surface, the second outer surface having a second central opening, wherein the first outer surface and the second outer surface face away from each other and the first inner surface and the second outer surface face toward each other;
a support member interconnecting the first and second flanges, the support member positioned transversely with respect to the first and second flanges; and
wherein the first flange, the second flange, and the support member are integrally formed with one another.

2. The I-beam spacer of claim 1, wherein the first and second flanges have substantially circular shapes.

3. The I-beam spacer of claim 1, wherein the first and second outer surfaces include a plurality of projections protruding outwardly therefrom.

4. The I-beam spacer of claim 1, wherein the second flange includes an arc-shaped opening following a curvature of the second flange.

5. The I-beam spacer of claim 1, wherein the support member has a circular cross-section along a portion of a length thereof.

6. The I-beam spacer of claim 1, wherein the support member has opening extending across a width thereof.

7. The I-beam spacer of claim 1, wherein the first outer surface includes a proximal portion and a distal portion, the proximal portion defining a proximal angle relative to the first inner surface.

8. The I-beam spacer of claim 7, wherein the distal portion of the first outer surface defines a distal angle relative to the first inner surface.

9. The I-beam spacer of claim 8, wherein the proximal and distal portions of the first outer surface meet at an apex.

10. The I-beam spacer of claim 1, wherein the first outer surface includes a plurality of holes extending along a length thereof.

11. The I-beam spacer of claim 10, wherein the holes are disposed in a linear arrangement.

12. The I-beam spacer of claim 1, wherein the support member includes a tapered distal end.

13. The I-beam spacer of claim 1, further comprising a convex structure positioned at a distal end of the I-beam spacer.

14. The I-beam spacer of claim 1, wherein the I-beam spacer has a curved profile.

15. An intervertebral fusion kit, comprising:
an I-beam spacer, which includes:
a first flange including a first outer surface and a first inner surface;
a second flange including a second outer surface and a second inner surface, wherein the first outer surface and the second outer surface face away from each other and the first inner surface and the second outer surface face toward each other;
a support member interconnecting the first and second flanges, the support member positioned transversely with respect to the first and second flanges; and
wherein the first flange, the second flange, and the support member are integrally formed with one another; and
a driver having a holding member at a distal end thereof, the holding member being adapted to hold the I-beam spacer.

16. A method for conducting intervertebral fusion, comprising:
providing an I-beam spacer for intervertebral fusion, which includes:
a first flange including a first outer surface and a first inner surface;
a second flange including a second outer surface and a second inner surface, the second outer surface having a second central opening, wherein the first outer surface and the second outer surface face away from each other and the first inner surface and the second outer surface face toward each other;
a support member interconnecting the first and second flanges, the support member positioned transversely with respect to the first and second flanges; and
wherein the first flange, the second flange, and the support member are integrally formed with one another;
making a recess between intervertebral bodies;
positioning the I-beam spacer in the recess; and
packing the recess with bone support matrix.
